(19) United States
(12) Patent Application Publication
(10) Pub. No.: US 2008/0045924 A1
(43) Pub. Date: Feb. 21, 2008

(54) Sonohysterography and biopsy catheter

(75) Inventors: Ted R. Cox, Winfield, AL (US); James M. Shwayder, Denver, CO (US); Benjamin T. Biltz, Bloomington, IN (US); John H. Ward, Spencer, IN (US); Daniel K. Whetham, Bloomington, IN (US); Troy W. Wingler, Martinsville, IN (US)

Correspondence Address: BRINKS HOFER GILSON & LIONE/CHICAGO/COOK, PO BOX 10395, CHICAGO, IL, 60610, US

(73) Assignees: Vance Products Incorporated, Spencer, IN (US); d/b/a Cook Urological Incorporated, Spencer, IN (US)

(21) Appl. No.: 11/729,633

(22) Filed: Mar. 29, 2007

Related U.S. Application Data
(60) Provisional application No. 60/787,760, filed on Mar. 31, 2006.

(51) Int. Cl. A61M 31/00

(52) U.S. Cl. 604/515

(57) ABSTRACT

A single device is provided for performing the diagnostic procedures of sonohysterography and endometrial biopsy. The device includes a cervical seal to occlude the cervix and a substance delivery and removal device to deliver and remove a fluid from the uterus. The substance delivery and removal device may also be used to create a vacuum so that suction occurs near side-ports that are used to collect endometrial tissue samples from multiple locations as the device is rotated or moved laterally within the patient. Additionally, the sample may be taken using a cellular collection device.

### RELATED APPLICATIONS

The present patent document claims the benefit of the filing date under 35 U.S.C. § 119(e) of Provisional U.S. Patent Application Ser. No. 60/787,760, filed Mar. 31, 2006, which is hereby incorporated by reference in its entirety.

### FIELD OF THE INVENTION

This invention relates to the diagnosis of uterine abnormalities, such as abnormal uterine bleeding and for performing diagnostic procedures for a uterus.

### BACKGROUND OF THE INVENTION

Medical professionals often use two procedures in order to diagnose abnormal uterine bleeding. The first procedure is sonohysterography, a technique employed for imaging the uterine cavity. Sonohysterography is commonly performed using a Goldstein catheter which is described in U.S. Pat. No. 6,706,026, assigned to the assignee of the present invention, and which is hereby incorporated in its entirety. Sonohysterography is performed by threading a catheter transcervically into the uterine cavity and delivering saline into the uterine cavity. The infusion of saline into the uterine cavity distends the cavity to provide contrast to the lining of the uterus. Diagnosis of endometrial and uterine pathology is performed by ultrasound.

If the cause of abnormal uterine bleeding is not determined via the sonohysterography, a second procedure is employed. The second procedure includes obtaining a biopsy of the endometrium to determine if hyperplasia exists. The endometrial biopsy is performed by using a device different from that used to perform the sonohysterography. Some medical professionals perform the endometrial biopsy using a pipelle such as the “Pipelle de Cornier” Endometrial Suction Curette produced by CooperSurgical, Inc., of Trumbull, Connecticut. The pipelle employs a ram-rod to collect the biopsy tissue sample. When the ram-rod is extracted it creates a vacuum which pulls tissue from the endometrial wall. Sufficient tissue is then collected for diagnosis.

The use of multiple devices to perform sonohysterography and endometrial biopsy may increase cramping of the uterus, patient pain, and patient discomfort. The use of multiple devices may also increase the amount of time necessary to complete the procedures, increase patient recovery time, increase the risk of injury to the patient, and may involve greater expense. It would be a great advantage if sonohysterography and endometrial biopsy will be performed using one device.

### BRIEF SUMMARY OF THE INVENTION

A device is provided for performing sonohysterography and endometrial biopsy, the device including an elongated tubular body having a proximal portion and a distal portion; at least one lumen extending throughout the elongated tubular body; a cervical seal located on the elongated tubular body; at least one side-port located on the distal portion of the elongated tubular body; and a syringe for communication with the elongated tubular body.

Further, a device is provided for performing sonohysterography and endometrial biopsy, the device including an elongated tubular body having a proximal portion and a distal portion; at least one lumen extending throughout the elongated tubular body; a female luer lock adapter attached to the proximal portion of the elongated tubular body; a cervical seal located on the elongated tubular body; at least two side-ports located on the distal portion of the elongated tubular body; and a syringe for connection to the female luer lock adapter.

Still further, a device is provided for performing sonohysterography and endometrial biopsy, the device including an elongated tubular body having a proximal portion and a distal portion; at least one lumen extending throughout the elongated tubular body; a cervical seal located on the elongated tubular body; at least one side-port located on the distal portion of the elongated tubular body; and a contrast fluid (substance) delivery and removal device for communication with the elongated tubular body.

A method also is provided for diagnosing uterine health, the method including inserting a device for performing sonohysterography and endometrial biopsy transcervically into a uterus; occluding a cervix using a cervical seal of the device; delivering an image enhancing medium into the uterus from a first syringe of the device; diagnosing the uterus using an electronic diagnostic tool; removing the image enhancing medium using the first syringe of the device; positioning the device for taking an endometrial biopsy; rotating at least a portion of the device to aspirate a sample of the endometrial biopsy; and collecting the sample by creating a vacuum using a second syringe of the device.

Further, a medical device is provided. The device includes an elongated tubular body having a proximal portion and a distal portion, a cervical seal located on the elongated tubular body, at least one fluid opening located at the distal portion of the elongated tubular body, at least one lumen extending between the proximal portion and the at least one fluid opening, and a substance delivery and removal device in communication with the at least one lumen.

In addition, a medical device is provided. The device includes an elongated tubular body having a proximal portion and a distal portion, a female luer lock adapter attached to the proximal portion of the elongated tubular body, and a cervical seal located on the elongated tubular body. The device further includes at least two side-ports located on the distal portion of the elongated tubular body, at least one lumen extending between the proximal portion and the at least two side-ports, and a substance delivery and removal device in connection with the female luer lock adapter.

Still further a medical device is provided. The device includes an elongated tubular body having a proximal portion and a distal portion, at least one fluid opening located at the distal portion of the elongated tubular body, at least one lumen extending between the proximal portion and the at least one fluid opening, a cellular collection device in communication with the elongated tubular body, an access sheath in communication with the elongated tubular body, and a cervical seal located on the access sheath.

Further, a method for diagnosing uterine health is provided. The method includes inserting a device for performing sonohysterography and endometrial biopsy transcervically into a uterus and occluding a cervix using a cervical seal of the device. The method further includes delivering an image enhancing medium into the uterus from a first substance delivery and removal device of the device, diagnosing the uterus using an electronic diagnostic tool, and removing the image enhancing medium using the first substance delivery and removal device of the device. The method further includes positioning the device for taking an endometrial biopsy, taking a sample of the endometrial biopsy, and collecting the sample by creating a vacuum using a second substance delivery and removal device of the device. The first and second substance delivery and removal devices are the same or different.

### BRIEF DESCRIPTION OF THE SEVERAL VIEWS OF THE DRAWINGS

The embodiments will be further described in connection with the attached drawing figures. It is intended that the drawings included as a part of this specification be illustrative of the embodiments and should in no way be considered as a limitation on the scope of the invention.

FIG. 1 is a plan view of an embodiment of the device;

FIGS. 1A-1I are partial plan views of the distal portion of embodiments of the device;

FIGS. 2A-2B are a partial cross-sectional views of embodiments of the device;

FIG. 3 is a schematic front view of the intrauterine cavity depicting a use of the device;

FIG. 3A is a schematic front view of a patient depicting a use of the device;

FIG. 4 is another schematic front view of the intrauterine cavity depicting a use of the device;

FIG. 5 is a partial cross-sectional view of another embodiment of the device;

FIG. 6 is schematic side-view of the endometrial wall depicting a use of the device;

FIG. 7 is flow-chart depicting a method of use of the device;

FIG. 8 is another partial cross-sectional view of an embodiment of the device;

FIG. 8A is a modified perspective view of an embodiment along the line 8A of FIG. 8;

FIG. 9 is another partial cross-sectional view of an embodiment of the device;

FIG. 10A is another partial cross-sectional view of an embodiment of the device;

FIG. 10B is a partial cross-sectional view of the proximal portion of another embodiment of the device;

FIG. 11 is another partial cross-sectional view of an embodiment of the device;

FIG. 12 is another partial cross-sectional view of an embodiment of the device;

FIG. 13 is another partial cross-sectional view of an embodiment of the device; and

FIG. 14 is another partial cross-sectional view of an embodiment of the device.

### DETAILED DESCRIPTION OF PRESENTLY PREFERRED EMBODIMENTS

The device described below provides a way to occlude the cervix, to deliver and remove an image enhancing fluid into and from the uterus, and to take a biopsy of the endometrium. Embodiments of the device provide an effective and safe procedure for performing a sonohysterography and an endometrial biopsy. The embodiments are particularly useful for diagnosing abnormal uterine bleeding, such as that associated with hyperplasia. The embodiments are not limited for use with a human.

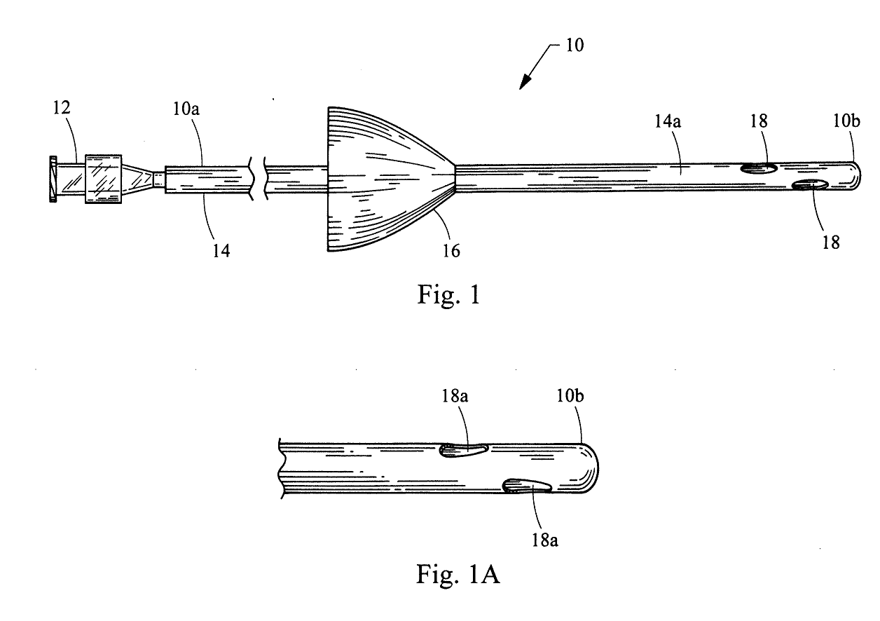

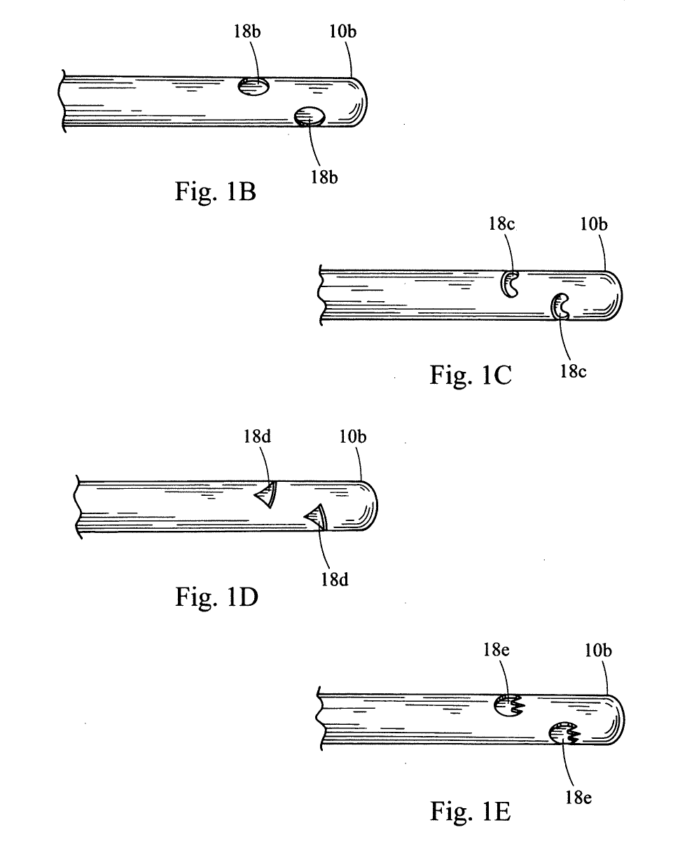

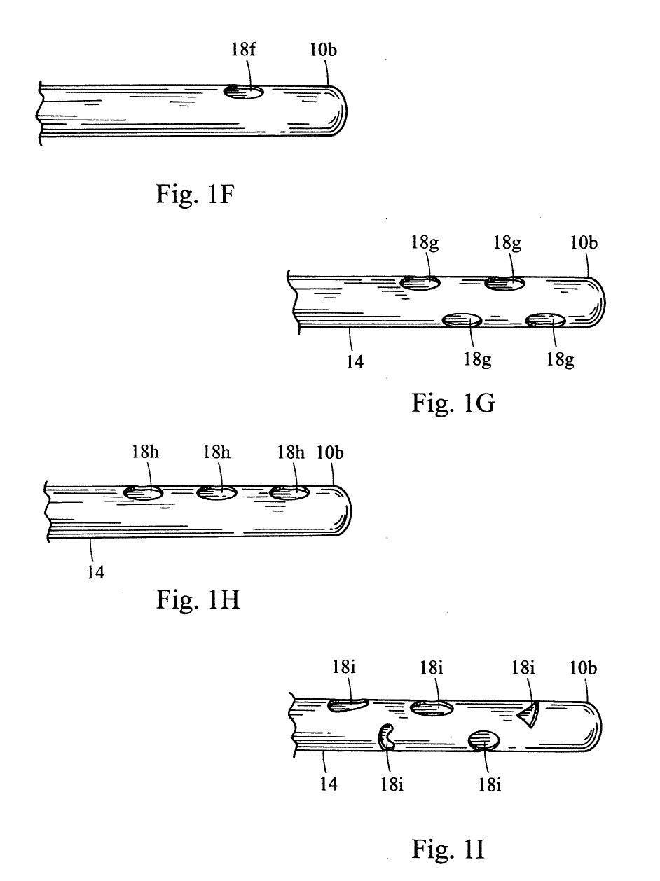

A more detailed description of the embodiments will now be given with reference to FIGS. 1-14. The present invention is not limited to those embodiments illustrated; it specifically contemplates other embodiments not illustrated and described but intended to be included in the claims. FIG. 1 is a plan view of a first embodiment of the device. A catheter assembly 10 has a proximal portion 10a, a distal portion 10b, and an elongated tubular body 14 having a lumen 14a extending throughout. Located at proximal portion 10a of catheter assembly 10 is a female luer lock adapter 12 which is connected to elongated tubular body 14. A cervical seal 16 for occluding the cervix is located on elongated tubular body 14. Located at distal portion 10b of catheter assembly are two side-ports 18 fluid openings located on opposite sides of elongated tubular body 14. Catheter assembly 10 is 26 cm long, although other lengths may be used. Cervical seal 16 is shaped like an acorn and is made from silicone, although other shapes may be used and other medically acceptable materials may be used. Cervical seals are described in U.S. Pat. No. 6,706,026, assigned to the assignee of the present application, and incorporated by reference herein.

Elongated tubular body 14 is preferably made from polytetrafluoroethylene (PTFE), although other medically accepted materials may be used such as polypropylene, polyurethane, or other Teflon-like materials. Teflon is marketed by E.I. duPont deNemours & Co., Wilmington, Del. Elongated tubular body 14 has a diameter of 7-9 Fr., although other sizes may be used. Side-ports 18 are located near distal portion Sob of catheter assembly 10 and are openings connected with lumen 14a. Side-ports 18 are used for delivering a fluid, such as a medically acceptable image enhancing medium including but not limited to saline, into the uterus. Side-ports 18 are also used for collecting a sample of the endometrium. The shape of side-ports 18 is oval with a length of 1-3 mm, as depicted in FIG. 1, although other shapes are contemplated.

Side-ports 18 may have a number of different shapes including, but not limited to, those shapes depicted in FIGS. 1A-1E. FIG. 1A depicts teardrop-shaped side-ports 18a that are 3 mm long, although other lengths may be used. FIG. 1B depicts round side-ports 18b that have a 2 mm diameter, although other diameters may be used. FIG. 1C depicts crescent-shaped side-ports 18c that are 3 mm in maximum dimension, although other dimensions may be used. FIG. 1D depicts diamond/wedge/triangular-shaped side-ports 18d that are 3 mm in maximum dimension, although other dimensions may be used. FIG. 1E depicts saw-tooth-shaped side-ports 18e that are 3 mm in maximum dimension, although other dimensions may be used. As shown in FIG. 1F, there is at least one side-port 18f located in elongated tubular body 14. There are two or more side-ports that are situated on opposite sides of elongated tubular body 14 so as to be able to collect endometrial samples from multiple locations (as depicted in FIG. 1).

Other configurations of side-ports 18 may be used including, but not limited to, those provided in FIGS. 1G-1I. FIG. 1G depicts four side-ports 18g located in opposite sides of elongated tubular body 14. FIG. 1H depicts another configuration wherein three side-ports 18h are located in-line in elongated tubular body 14. FIG. 1I depicts yet another configuration wherein the five side-ports 18i each have different shapes and are located along the length of elongated tubular body 14.

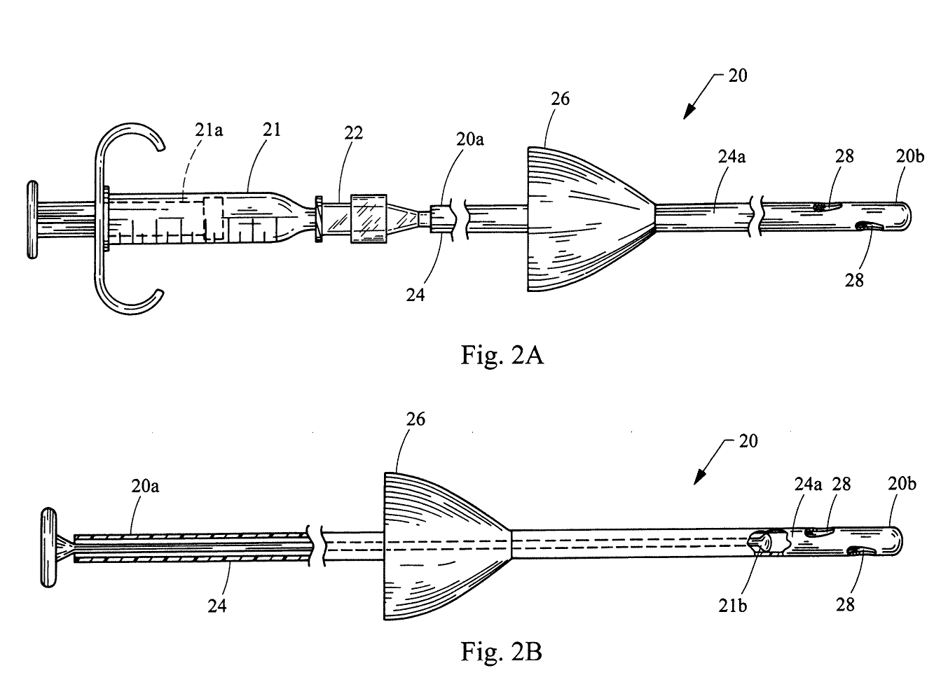

Further embodiments are shown in FIGS. 2-10B. FIG. 2A is a partial cross-sectional view of an embodiment of the device. A catheter assembly 20 includes an elongated tubular body 24 and has a proximal portion 20a and a distal portion 20b. Connected to elongated tubular body 24 is a female luer lock adapter 22 which accepts a luer lock or luer-slip syringe 21. Other types of connectors are contemplated. Located on elongated tubular body 24 is a cervical seal 26 which is used to occlude the cervix. Elongated tubular body 24 contains a lumen 24a extending throughout wherein fluid may be instilled or tissue removed under suction by syringe 21, a substance delivery and removal device. Located at distal portion 20b of catheter assembly 20 are side-ports 28 that are teardrop-shaped. As syringe plunger 21a is actuated upon removal, it creates a vacuum within lumen 24a with resulting suction at side-ports 28. The suction at side-ports 28 pulls and removes endometrial tissue cells from multiple locations as side-ports 28 contact the endometrium and catheter assembly 20 rotates within the uterus. The sample is then analyzed.

FIG. 2B is another a partial cross-sectional view of an embodiment of the device. A catheter assembly 20 is shown without syringe 21 or female luer lock adapter 22 as shown in FIG. 2A. Instead, a plunger substance delivery and removal device 21b is inserted directly into lumen 24a of elongated tubular body 24. As plunger 21b is removed, it creates a vacuum within lumen 24a of elongated tubular body 24 which creates suction at side-ports 28. The suction at side-ports 28 removes endometrial tissue cells from multiple locations as side-ports 28 contact the endometrium and catheter assembly 20 rotates within the uterus. The sample is then analyzed.

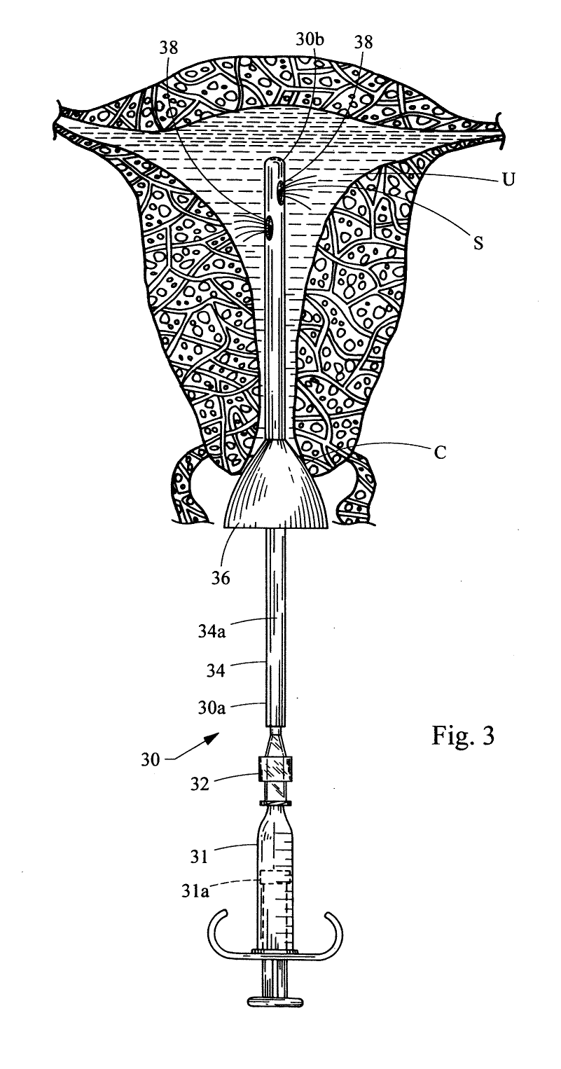

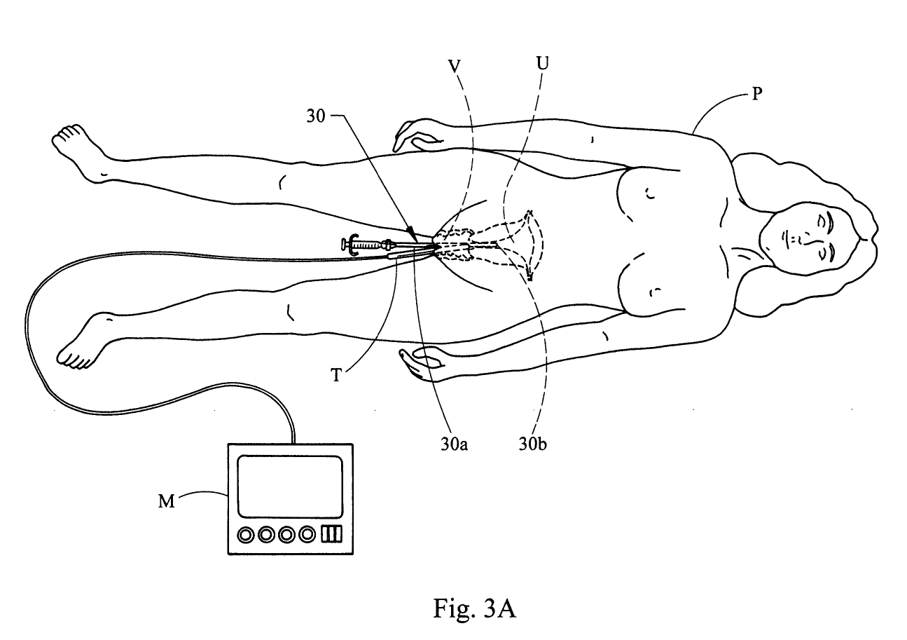

FIG. 3 depicts a schematic front view of the intrauterine cavity depicting a use of the device. A catheter assembly 30 includes an elongated tubular body 34, a proximal portion 30a, and a distal portion 30b. Elongated tubular body 34 has a lumen 34a extending throughout. Located at proximal portion 30a of catheter assembly 30 is a female luer lock adapter 32 which accepts a luer lock or luer-slip syringe 31. A cervical seal 36, used to occlude the cervix C, is located on elongated tubular body 34. At distal portion 30b of catheter assembly 30 are oval-shaped side-ports 38. Distal portion 30b of catheter assembly 30 is shown in the uterus U after transcervical placement. Cervix C is occluded by cervical seal 36 which is acorn-shaped, although other shapes may be used.

To perform the sonohysterography, 5-10 ml (other amounts may be used) of saline S, or other medically acceptable image contrast fluid, is delivered from syringe 31 through side-ports 38. After the sonohysterography is performed, saline S is removed from uterus U by pulling back on the plunger 31a of syringe 31 to create light suction.

FIG. 3A depicts a schematic front view of a patient depicting a use of catheter assembly 30. Once saline S is delivered to uterus U, as depicted in FIG. 3, an ultrasound electronic diagnostic tool is used to diagnose uterine health, as depicted in FIG. 3A. Electronic diagnostic tools other than an ultrasound system may also be used to diagnose uterine health. These tools include, but are not limited to, an x-ray system, ultraviolet light system, or fluoroscopy system. The patient P, not limited to a human being, is shown with the proximal portion 30a of catheter assembly 30 extending out from the vagina V after catheter assembly 30 was transcervically placed into the uterus U of patient P. Thus, distal portion 30b of catheter assembly is shown within uterus U. An ultrasonic transducer T is used to generate images of endometrial and uterine pathology. In order to determine the cause of abnormal uterine bleeding or other uterine abnormality, the images of endometrial and uterine pathology are viewed on the ultrasonic image monitor M which is connected to ultrasonic transducer T. After an image is read using ultrasonic image monitor M, plunger 31a is actuated to create light suction in order to remove saline S from uterus U (as depicted in FIG. 3).

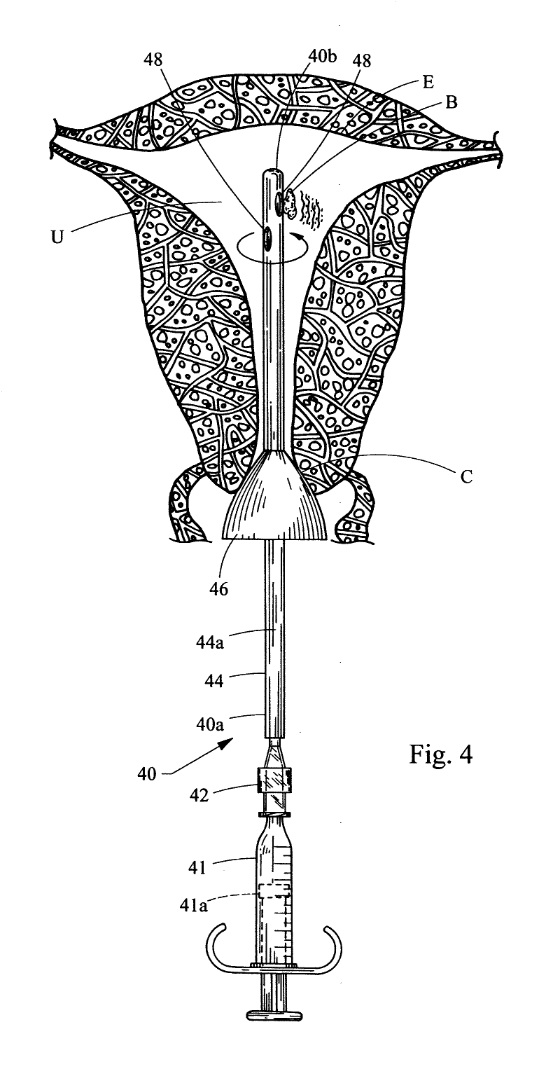

FIG. 4 is another schematic front view of the intrauterine cavity depicting a use of the device. A catheter assembly 40 has a proximal portion 40a, a distal portion 40b, and includes an elongated tubular body 44 having a lumen extending throughout 44a, and is attached to a female luer lock adapter 42. Located on elongated tubular body 44 is a cervical seal 46 that is acorn-shaped, although other shapes may be used. Located at distal portion 40b of catheter assembly 40 are side-ports 48 that are oval-shaped. Distal portion 40b of catheter assembly 40 is depicted in the uterus U after transcervical placement. Cervical seal 46 is occluding the cervix C. Catheter assembly 40 is rotated so that side-ports 48 may contact endometrial tissue E of uterus U from multiple locations. The endometrial tissue sample B is collected from uterus U by actuating the plunger 41a of syringe 41. Actuating plunger 41a creates light suction at side-ports 48. This suction creates a vacuum within lumen 44a of elongated tubular body 44 and, in turn, creates suction near side-ports 48. The suction at side-ports 48 pulls endometrial tissue E off uterus U to be collected as an endometrial tissue sample B. Endometrial tissue sample B is then analyzed.

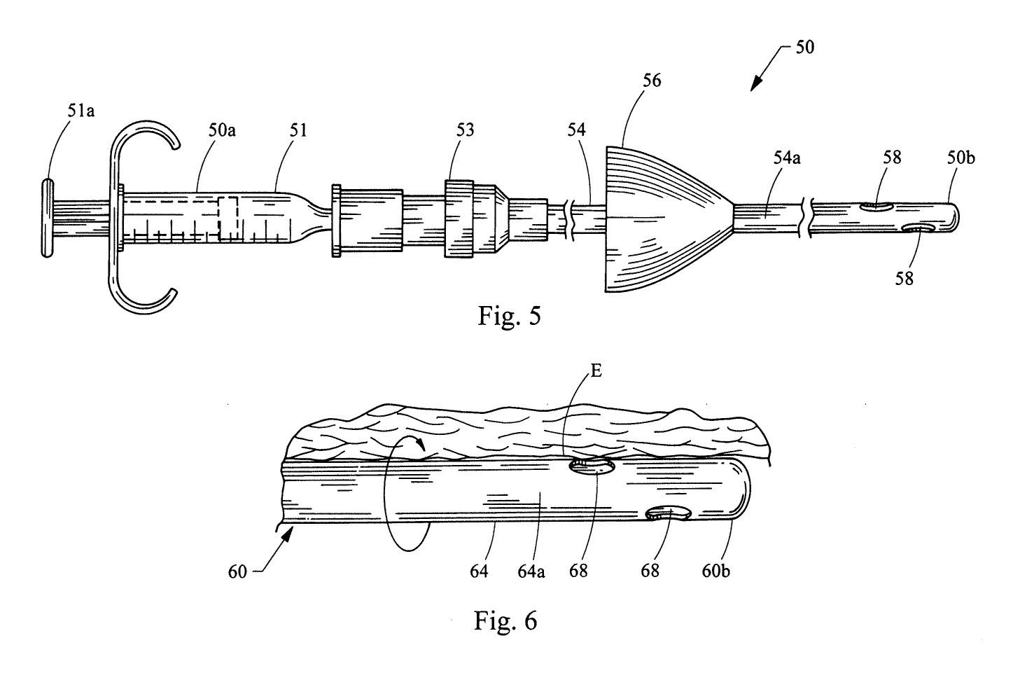

FIG. 5 is a partial cross-sectional view of another embodiment of the device. This embodiment of a catheter assembly 50 has a proximal portion 50a, a distal portion 50b, and an elongated tubular body 54 having a lumen 54a extending throughout. The elongated tubular body 54 is attached to a rotatable fitting 53. Located on elongated tubular body 54 is a slideable cervical seal 56 which is acorn-shaped. At distal portion 50b of catheter assembly are side-ports 58 which are oval-shaped although a different number and shape of side ports is contemplated. Rotatable fitting 53 rotates distal portion 50b of catheter assembly 50 to aid in the collection of tissue samples from multiple locations. Rotatable fitting 53 is engaged so that distal portion 50b of catheter assembly 50 rotates to allow for the capture of endometrial samples from multiple locations. Light suction is created by pulling back on the plunger 51a of syringe 51 to create a vacuum within lumen 54a of elongated tubular body 54 with resulting suction at side-ports 58. As distal portion 50b of catheter assembly 50 is rotated, side-ports 54 contact the endometrial tissue wall of the uterus and the vacuum aspirates the sample. Rotatable fitting 53 provides the medical professional the ability to rotate distal portion 50b of catheter assembly 50 without having to rotate proximal portion 50a of catheter assembly 50. Further details of rotatable fitting 53 are described in U.S. Patent Application Pub. No. 2005/0137500 that is hereby incorporated by reference in its entirety.

FIG. 6 depicts the distal portion 60b of a portion of catheter assembly 60. Shown in the figure is a portion of the elongated tubular body 64 having a lumen 64a extending throughout. Located on elongated tubular body 64 are side-ports 68. Light suction is applied to lumen 64a within elongated tubular body 64, and resulting vacuum is created near side-ports 68. As catheter assembly 60 is rotated, side-ports 68 come into contact with endometrial tissue E from multiple locations. The vacuum creates suction at side-ports 68 which collect endometrial tissue E for analysis from the multiple locations that side-ports 68 contact.

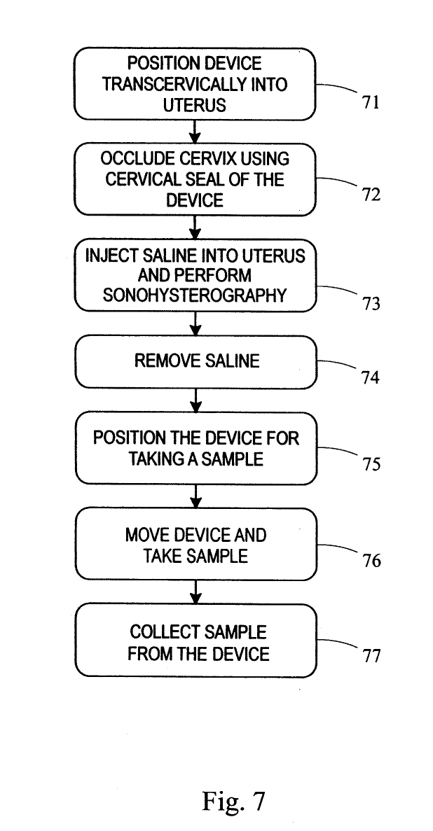

FIG. 7 depicts a method of using an embodiment. The device is positioned transcervically into the uterus 71, and the cervix is occluded using a cervical seal of the device 72. Saline (or another fluid) is injected into the uterus and the sonohysterography is performed 73. The fluid is then removed 74. The device is then positioned for taking a sample 75. The position of the device can be determined based on the sonohysterography results. For example, if the sonohysterography reveals a lesion, the device can be positioned for taking an endometrial biopsy at the location of the lesion. The device is then rotated or moved laterally, and a sample is taken by aspiration or by rubbing a cellular collection device, including but not limited to a brush assembly, sponge, cloth, or other type of porous material onto the endometrial wall 76. Additionally, the device need not be moved depending upon the patient's needs and the area of the uterus from which the sample need be collected. Finally, the sample is collected from the device 77. The collection of the sample is completed extra-corporeally upon device removal by ejecting the sample from the device into a specimen cup or by following any other medically acceptable method for collecting a sample.

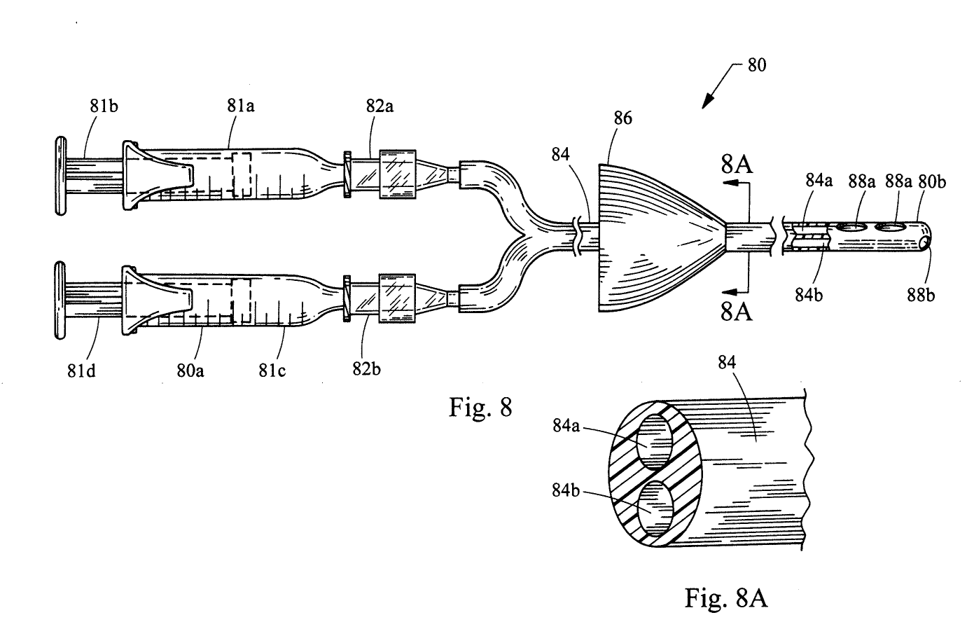

Referring to FIG. 8 and FIG. 8A, FIG. 8 depicts another partial cross-sectional view of an embodiment of the device, and FIG. 8A depicts a modified perspective view of an embodiment along the line 8A of FIG. 8. A catheter assembly 80 has a proximal portion 80a, a distal portion 80b, and an elongated tubular body 84 with a tissue lumen 84a and a fluid lumen 84b extending throughout. Located at proximal portion 80a of catheter assembly 80 are two female luer lock adapters 82a and 82b. Female luer lock adapter 82a is connected to tissue lumen 84a. Connected to female luer lock adapter 82a is syringe 81a, having a plunger 81b. Located on tissue lumen 84a are side-ports 88a. Connected to fluid lumen 84b is a female luer lock adapter 82b which is connected to a syringe 81c having a plunger 81d. Located in fluid lumen 84b is a fluid port 88b, which can also be located on the side of elongated tubular body 84. Catheter assembly 80 is placed transcervically into the uterus. A slideable cervical seal 86 is used to occlude the cervix. A fluid such as saline or any other medically acceptable image enhancing fluid, is delivered from syringe 81c and out through fluid port 88b into the uterus. The sonohysterography is performed, after which, the saline is removed using syringe 81c. The endometrial biopsy is then performed. Catheter assembly 80 is rotated so that side-ports 88a may contact the endometrial tissue of the uterus from multiple locations. The endometrial tissue sample is collected from the uterus by withdrawing plunger 81b of syringe 81a so that a vacuum is created within tissue lumen 84a of elongated tubular body 84 which in turn creates suction near side-ports 88a. The suction at side-ports 88a pulls the endometrial tissue off the uterus so that it can be analyzed.

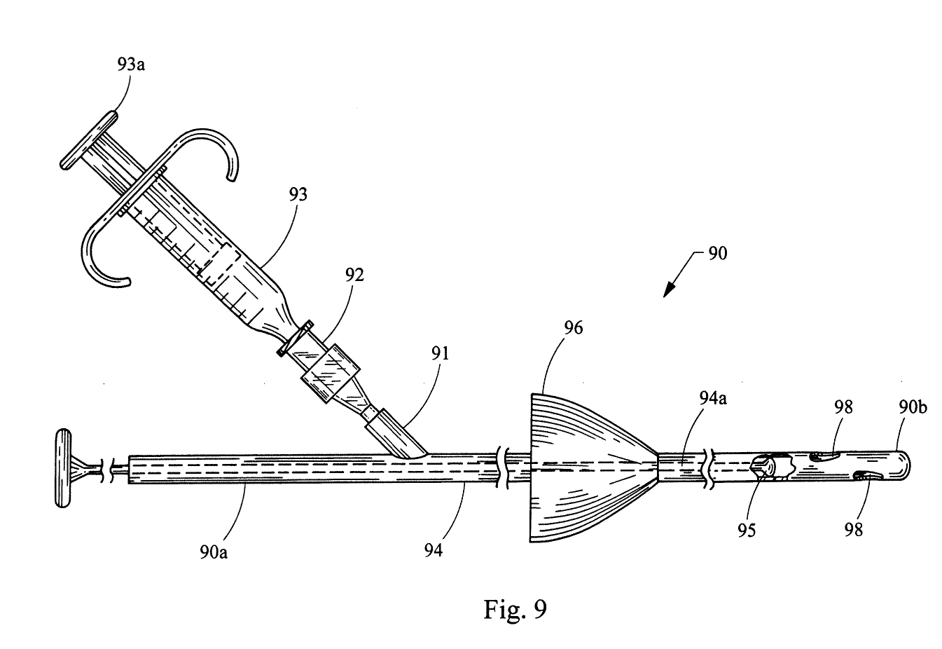

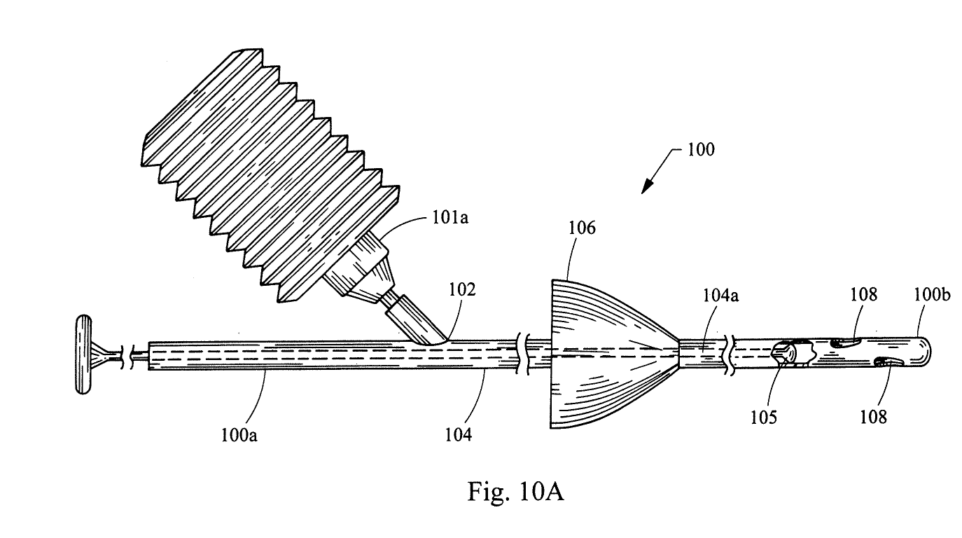

FIG. 9 is another partial cross-sectional view of an embodiment of the device which depicts a catheter assembly 90 having an elongated tubular body 94 having a proximal portion 90a and a distal portion 90b. Located on elongated tubular body 94 are a cervical seal 96 and two side-ports 98. At proximal portion 90a is a syringe port 91 having an attached female luer lock adapter 92. Attached to female luer lock adapter 92 is syringe 93 having a plunger 93a. Located within lumen 94a is another plunger 95. To perform the sonohysterography, catheter assembly 90 is transcervically placed into the uterus. Syringe 93 delivers saline into the uterus through side-ports 98, and the sonohysterography is performed. Saline is removed from the uterus by actuating plunger 93a of syringe 93. The endometrial biopsy is performed by pushing plunger 95 past syringe port 91. Plunger 95 is then actuated which creates a vacuum within lumen 94a, which in turn creates suction at side-ports 98. As catheter assembly 90 is rotated, side-ports 98 come into contact with endometrial tissue from multiple locations. The suction at side-ports 98 collects endometrial tissue from the multiple locations that side-ports 98 contact. The sample is then analyzed.

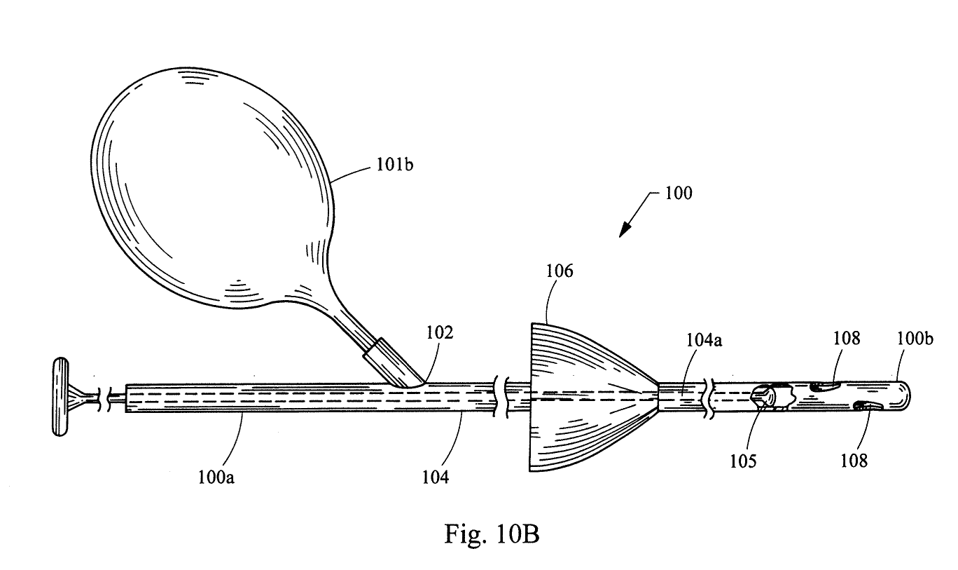

FIGS. 10A and 10B depict partial cross-sectional views of embodiments of the device in which catheter assembly 100 has an elongated tubular body 104 having a proximal portion 100a and a distal portion 10b. At distal portion 100b are side-ports 108 and a cervical seal 106. Located at proximal portion 100a is a substance delivery and removal device accordion-bulb 101a, which is attached at port 102 to elongated tubular body 104. Within lumen 104a of elongated tubular body is a plunger 105. To perform the sonohysterography, catheter assembly 100 is transcervically placed into the uterus. Accordion bulb 101a, pre-filled with a fluid such as saline, is squeezed to deliver saline and the sonohysterography is performed. Saline is removed from the uterus by squeezing accordion bulb 101a which creates a vacuum within lumen 104a and pulls the saline back into the accordion bulb 101a.

The endometrial biopsy is performed by pushing plunger 105 past side-port 102. Plunger 105 is then removed creating a vacuum within lumen 104a, which in turn creates suction at side-ports 108. As catheter assembly 100 is rotated, side-ports 108 come into contact with endometrial tissue from multiple locations. The suction at side-ports 108 collects endometrial tissue from the multiple locations that side-ports 108 contact.

FIG. 10B is a partial cross-sectional view of the proximal portion of another embodiment of the device. Accordion bulb 101a (as shown in FIG. 10A) is replaced with squeezable substance delivery and removal device oval-bulb 101b. The shape of the saline-delivery tool is not limited to that of an accordion 101a (as shown in FIG. 10A) or bulb 101b (as shown in FIG. 10B).

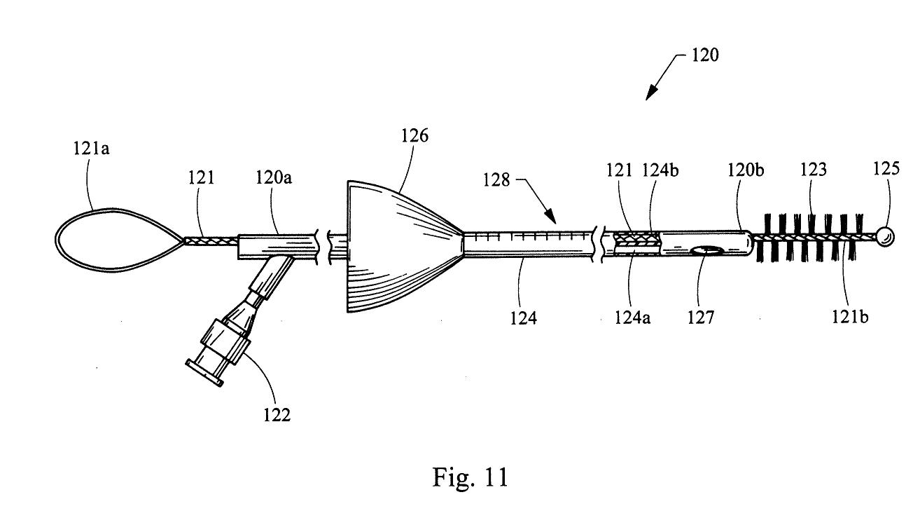

FIG. 11 is a partial cross-sectional view of another embodiment of the device. This embodiment of a catheter assembly 120 has a proximal portion 120a, a distal portion 120b, and an elongated tubular body 124 having two lumens 124a 124b disposed within. Elongated tubular body 124 is about 9 Fr. and 26 cm long, however, other sizes are contemplated depending upon the bodily part sought and the needs of the patient. As with all the embodiments described herein, ink marks, radiopaque bands, and/or other distance and placement markings or indicators may be disposed on or about the catheter. For example, ink marks 128 are placed at 1 cm intervals to 10 cm. Other distances are contemplated. Optional cervical seal 126 is used to occlude the cervix.

First lumen, 124a is a substance delivery and removal lumen that stretches from proximal portion 120a of catheter to side-port 127 located on elongated tubular body 124. Side-port 127 is not limited to the shape or number depicted in FIG. 11—other shapes and numbers of side-port are contemplated including those discussed above. Additionally, it is contemplated that first lumen could stretch from proximal portion 120a to distal portion 120b. Second lumen 124b stretches from proximal portion 120a to distal portion 120b. In communication with first lumen 124a is a luer lock adapter 122 that accepts a luer lock or luer-slip substance delivery and removal device, such as a syringe (not shown). Although shown having a luer lock adapter 122, other connections are contemplated such as those previously depicted, described, and known in the art. Additionally, any of the previously depicted and described substance delivery and removal devices are also contemplated for use with this embodiment as are those known in the art.

Fluid may be instilled using a syringe (not shown) or any other substance delivery and removal device attached to luer lock 122 as a means for delivering and removing a substance, such as a radiopaque fluid including but not limited to saline, in order to perform a medical procedure such as a sonohysterography. The saline can then be removed by using a substance removal device such a syringe.

The biopsy is performed using brush assembly 121. Brush assembly 121 is made from a nylon filament; however, other medically acceptable materials are contemplated. Brush assembly is further described in U.S. Pat. No. 5,713,369, entitled “Uterine Endometrial Tissue Sample Brush,” assigned to the assignee of the present application, and is hereby incorporated by reference. In addition, the TAO Brush I.U.M.C. Endometrial Sampler, further described on the World Wide Web at www.cookgroup.com/cook_obgyn/products/gynecology/2_{—}03/2_{—}03_{—}01.html, available from the assignee of the present application, and hereby incorporated by references, is contemplated to work well with this embodiment.

Bristles 123 of brush assembly 121 contact the uterine wall and remove cellular material therefrom. As bristles 123 pass over the cells, cellular material will adhere to bristles 123 and can be collected for further examination and lab work. The devices and methods are not, however, limited to use with a brush assembly. For example other cellular collection devices can be used including but not limited to sponges, cloths, or other types of porous materials that collect cellular material when put in contact with cells.

Distal portion 121b of brush assembly 121 is pushed into second lumen 124b so that it extends out through distal portion of elongated tubular body 124. Distal portion 121b of brush assembly 121 terminates with atraumatic ball structure 125 to prevent injury or damage to the uterus. Endometrial cells will adhere to bristles 123 as brush assembly 121 is moved laterally or radially such that they contact endometrial tissue. Brush assembly 121 can then be removed from elongated tubular body 124 to collect the sample by pulling proximal portion 121a of brush assembly 121 out from elongated tubular body 124. Proximal portion 121a of brush assembly 121 is not limited to having an open-circular shape; other shapes and structures are contemplated including but not limited to squares, rectangles, and other ergonomic designs.

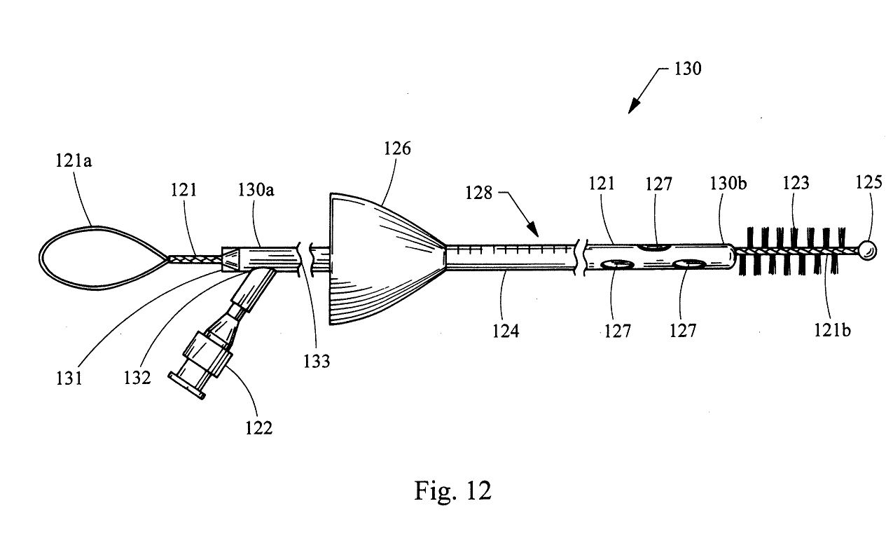

FIG. 12 is another partial cross-sectional view of an embodiment of the device. FIG. 12 is like that shown in FIG. 11; however, catheter assembly 130 contains only one lumen 133 extending from proximal portion 130a to distal portion 130b. At proximal portion 130 of catheter assembly 130, the device separates to a y-connection 132 such that brush assembly 121 travels through check flow valve 131 and syringe (not shown) can attach to luer lock connector 122. Check flow valve 131 prevents backflow of fluid traveling through lumen 133 out through where brush assembly 121 enters elongated tubular body 124.

Like the device shown in FIG. 12, brush assembly 121 is laterally and rotatably moveable within elongated tubular body 124 such that bristles 123 can dwell within elongated tubular body 124 while the device is being maneuvered in order to protect the patent from accidental bristle contact. This also prevents bristles 123 from being snagged or damaged. Once positioned, the sonohysterography can be performed by instilling a fluid, such as saline, using a substance delivery and removal device, such as a syringe (not shown), attached to connector 122. The fluid will pass out though side-ports 127 and can later be removed by aspiration. Use of more or less side-ports is contemplated.

The device may then need to be repositioned depending upon the results of the sonohysterography. Once positioned, brush assembly 121 is extended out from distal portion of elongated tubular body 124. Bristles 123 come in contact with endometrial tissue, and cellular material adheres to bristles 123. Brush assembly 121 can then be repositioned within elongated tubular body 124 and catheter assembly 130 can then be removed from the patient. As stated above, the device is not limited for use with brush assembly; other cellular collection devices are contemplated.

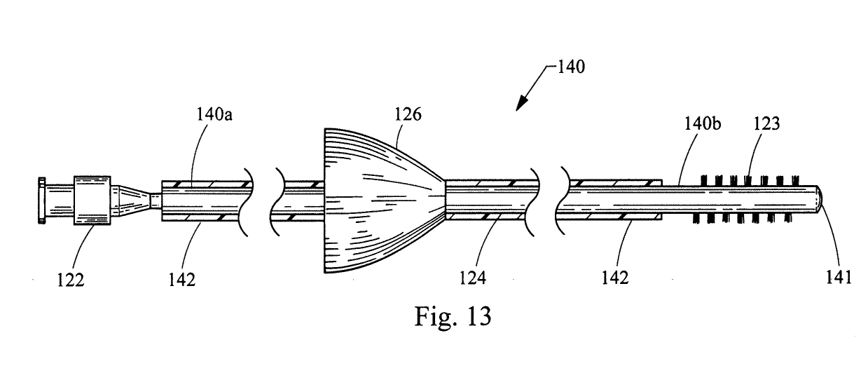

FIG. 13 depicts another partial cross-sectional view of an embodiment of the device. Catheter assembly 140 has proximal portion 140a and distal portion 140b and lumen 141 extending throughout. Bristles 123 are located directly on elongated tubular body 124. Bristles 123 are approximately 2-3 mm long and distributed about 2.5-3 cm back from distal portion of elongated tubular body 124. Other sizes and dimensions are contemplated depending upon the needs to the patient and the cellular material to be collected. Other cellular collection devices are also contemplated in being attached to elongated tubular body, such as those described above.

Elongated tubular body 124 is laterally and rotatably moveable within access sheath 142. Access sheath 142 is attached to cervical seal 126. As device is being positioned within the patient, bristles 123 are withdrawn within access sheath 142 such that bristles 123 are covered. This protects the patient from accidental contact with the bristles. It also protects the bristles from being snagged or damaged. Once positioned, the sonohysterography can be performed by instilling a fluid through connector 122 that will travel from lumen 141 and out the distal portion 140b of catheter assembly. The fluid can then be removed by aspiration.

Once the device is positioned to perform the biopsy, bristles 123 are advanced out from access sheath 142. The device is then moved laterally and/or rotationally depending upon the needs of the patient in order to obtain a cellular sample.

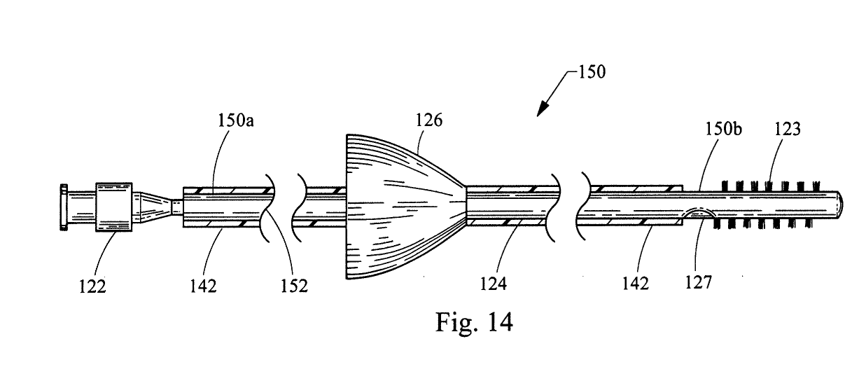

The embodiment depicted in FIG. 14 is similar to that depicted in FIG. 13. Catheter assembly 150 also contains access sheath 142 to cover bristles 123 as needed. Single lumen 152 extends from proximal portion 150a to side-port 127. Use of additional side-ports is contemplated. Fluid is expelled and recovered from side-port 127 as needed to perform the sonohysterography or any other procedure needing fluid. As with all the embodiments, the tips are atraumatic.

As is evident, the embodiments provide a very effective design for performing sonohysterography and endometrial biopsy. The embodiments are not limited to perform sonohysterography and endometrial biopsy. Instead, other types of biopsy are also contemplated. Moreover, other parts of the body that would benefit from irrigation would also likely benefit from the use of this device.

The foregoing description and drawings are provided for illustrative purposes only and are not intended to limit the scope of the invention described herein or with regard to the details of its construction and manner of operation. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic and descriptive sense only and not for the purpose of limiting the scope of the invention set forth in the following claims. Moreover the device is not limited to any specific dimension or material discussed above, nor is the device limited to being used with saline or an image contrast fluid alone.

1. A medical device comprising:
an elongated tubular body having a proximal portion and a distal portion;
a cervical seal located on the elongated tubular body;
at least one fluid opening located at the distal portion of the elongated tubular body;
at least one lumen extending between the proximal portion and the at least one fluid opening; and
a substance delivery and removal device in communication with the at least one lumen.

2. The device according to claim 1 further comprising a second lumen extending from the proximal portion to the distal portion of the elongated tubular body.

3. The device according to claim 1 further comprising a cellular collection device in communication with the elongated tubular body.

4. The device according to claim 1 wherein the cervical seal is acorn-shaped.

5. The device according to claim 1 wherein the at least one fluid opening has a shape selected from the group consisting of an oval, a teardrop, a circle, a saw-tooth, a crescent, a wedge, a diamond, and a triangle.

6. The device according to claim 1 wherein the device is about 26 cm long.

7. The device according to claim 1 further comprising a female luer lock adapter connected to the proximal portion of the elongated tubular body.

8. The device according to claim 1 further comprising a rotatable fitting connected to the substance delivery and removal device.

9. The device according to claim 1 wherein the at least one fluid opening comprises at least two side-ports located on opposite sides of the distal portion of the elongated tubular body.

10. The device according to claim 1 wherein the elongated tubular body is made from polyurethane, polytetrafluoroethylene, or polypropylene.

11. The device according to claim 1 wherein the cervical seal is able to slide along the elongated tubular body.

12. The device according to claim 1 wherein the elongated tubular body further comprises a second lumen and a second substance delivery and removal device in communication with the elongated tubular body.

13. The device according to claim 1 wherein the substance delivery and removal device is an accordion device, bulb, syringe, or plunger.

14. A medical device comprising:
an elongated tubular body having a proximal portion and a distal portion;
a female luer lock adapter attached to the proximal portion of the elongated tubular body;
a cervical seal located on the elongated tubular body;
at least two side-ports located on the distal portion of the elongated tubular body;
at least one lumen extending between the proximal portion and the at least two side-ports; and
a substance delivery and removal device in connection with the female luer lock adapter.

15. The device according to claim 14 further comprising a second lumen extending from the proximal portion to the distal portion of the elongated tubular body.

16. The device according to claim 15 further comprising a cellular collection device in communication with the elongated tubular body.

17. The device according to claim 14 further comprising a rotatable fitting connected to the proximal portion of the elongated tubular body.

18. The device according to claim 14 wherein the cervical seal is able to slide along the elongated tubular body.

19. A medical device comprising:
an elongated tubular body having a proximal portion and a distal portion;
at least one fluid opening located at the distal portion of the elongated tubular body;
at least one lumen extending between the proximal portion and the at least one fluid opening;
a cellular collection device in communication with the elongated tubular body;
an access sheath in communication with the elongated tubular body; and
a cervical seal located on the access sheath.

20. The device according to claim 19 further comprising a substance delivery and removal device.

21. The device according to claim 19 further comprising a second lumen extending between the proximal portion and the distal portion of the elongated tubular body.

22. A method for diagnosing uterine health, the method comprising:
inserting a device for performing sonohysterography and endometrial biopsy transcervically into a uterus;
occluding a cervix using a cervical seal of the device;
delivering an image enhancing medium into the uterus from a first substance delivery and removal device of the device;
diagnosing the uterus using an electronic diagnostic tool;
removing the image enhancing medium using the first substance delivery and removal device of the device;
positioning the device for taking an endometrial biopsy;
taking a sample of the endometrial biopsy;
collecting the sample; and
wherein the first and second substance delivery and removal devices are the same or different.

23. The method according to claim 22 wherein the cervix is occluded by using an acorn-shaped cervical seal of the device.

24. The method according to claim 22 wherein the image enhancing medium is saline.

25. The method according to claim 22 wherein the electronic diagnostic tool is selected from the group consisting of an ultrasound system, an x-ray system, an ultraviolet light system, and a fluoroscopy system.

26. The method according to claim 22 wherein the positioning of the device for taking an endometrial biopsy is determined based on the results from diagnosing the uterus using an electronic diagnostic tool.

27. The method according to claim 22 wherein the taking the sample is performed using a cellular collection device or aspiration.
